(19) United States
(12) Patent Application Publication
(10) Pub. No.: US 2016/0249884 A1
(43) Pub. Date: Sep. 1, 2016

(54) ULTRASONIC DIAGNOSTIC APPARATUS AND METHOD OF MEASURING ELASTICITY

(71) Applicant: General Electric Company, Schenectady, NY (US)

(72) Inventors: Hiroshi Hashimoto, Tokyo (JP); Shunichiro Tanigawa, Tokyo (JP)

(21) Appl. No.: 15/046,003

(22) Filed: Feb. 17, 2016

(30) Foreign Application Priority Data
Feb. 27, 2015 (JP) 2015-037520

(51) Int. Cl. A61B 8/08; A61B 8/00

(52) U.S. Cl. CPC A61B 8/485; A61B 8/54; A61B 8/5223; A61B 8/488; A61B 8/463

(57) ABSTRACT

An ultrasonic diagnostic apparatus and method includes controlling, with a transmission control section, a transmission of an ultrasonic push pulse to biological tissue in a subject. The apparatus and method includes controlling, with the transmission control section, transmission of an ultrasonic detecting pulse for detecting shear waves generated in said biological tissue by said push pulse. The apparatus and method includes calculating, with a measurement-value calculating section, a measurement value regarding elasticity of said biological tissue based on echo signals of said ultrasonic detecting pulse. The apparatus and method includes creating, with a Doppler processing section, Doppler data based on said echo signals of said ultrasonic detecting pulse. The apparatus and method includes displaying an image based on the Doppler data on a display section.

### CROSS-REFERENCE TO RELATED APPLICATIONS

This application claims priority to Japan patent application number 2015-037520, filed on Feb. 27, 2015, the entirety of which is incorporated herein by reference.

### BACKGROUND

The present disclosure relates to an ultrasonic diagnostic apparatus and method for transmitting an ultrasonic push pulse and measuring elasticity of biological tissue.

There have been known elasticity measurement techniques of measuring elasticity of biological tissue by transmitting an ultrasonic pulse (push pulse) having a high acoustic pressure from an ultrasonic probe to the biological tissue. More particularly, shear waves generated in the biological tissue by the push pulse are detected by ultrasonic detecting pulses, and the velocity of propagation of the shear waves and/or the elasticity value of the biological tissue are calculated to provide elasticity data. Then, an elasticity image having colors or the like depending upon the elasticity data is displayed.

The shear waves are detected in a two-dimensional region of interest defined by a user or the like. The elasticity data is then obtained and an elasticity image is displayed for the two-dimensional region of interest.

In the case that a blood vessel exists in the region of interest, there is a concern that sometimes an elasticity image accurately reflecting the elasticity of biological tissue cannot be displayed. Moreover, a user may want to confirm positional correspondence between the position and distribution of the blood vessel and a suspected lesion in an elasticity image. Accordingly, it is desired to provide an ultrasonic diagnostic apparatus and a program for controlling the same capable of displaying an image allowing confirmation of the presence of blood flow and an elasticity image without lowering the frame rate.

According to an embodiment, Doppler data is created in addition to the measurement value regarding elasticity of biological tissue based on echo signals of an ultrasonic detecting pulse for detecting shear waves, and therefore, an elasticity image based on the measurement value and a Doppler image based on the Doppler data can be displayed without lowering the frame rate.

### BRIEF SUMMARY OF INVENTION

In an embodiment, an ultrasonic diagnostic apparatus includes a transmission control section for controlling transmission of an ultrasonic push pulse to biological tissue in a subject, and transmission of an ultrasonic detecting pulse for detecting shear waves generated in said biological tissue by said push pulse, a measurement-value calculating section for calculating a measurement value regarding elasticity of said biological tissue based on echo signals of said ultrasonic detecting pulse, and a Doppler processing section for creating Doppler data based on said echo signals of said ultrasonic detecting pulse.

In an embodiment, a method of measuring elasticity with an ultrasonic diagnostic apparatus includes controlling, with a transmission control section, a transmission of an ultrasonic push pulse to biological tissue in a subject. The method includes controlling, with the transmission control section, transmission of an ultrasonic detecting pulse for detecting shear waves generated in said biological tissue by said push pulse. The method includes calculating, with a measurement-value calculating section, a measurement value regarding elasticity of said biological tissue based on echo signals of said ultrasonic detecting pulse. The method includes creating, with a Doppler processing section, Doppler data based on said echo signals of said ultrasonic detecting pulse. The method includes displaying an image based on the Doppler data on a display section.

### BRIEF DESCRIPTION OF THE DRAWINGS

FIG. 1 is a block diagram showing a schematic configuration of an ultrasonic diagnostic apparatus, which is an exemplary embodiment of the present invention.

FIG. 2 is a block diagram showing a configuration of an echo data processing section.

FIG. 3 is a block diagram showing a configuration of a display processing section.

FIG. 4 is a diagram showing a display section in which a B-mode image and a color Doppler image are displayed.

FIG. 5 is a diagram showing the display section in which a B-mode image, a color Doppler image, and an elasticity image are displayed.

FIG. 6 is a flow chart showing an operation of the embodiment.

FIG. 7 is a block diagram for explaining processing at Step S7 in the flow chart in FIG. 6.

FIG. 8 is an explanatory diagram showing echo data of ultrasonic detecting pulses.

FIG. 9 is an explanatory diagram showing a plurality of acoustic lines in a region of interest and one point on one of the plurality of acoustic lines.

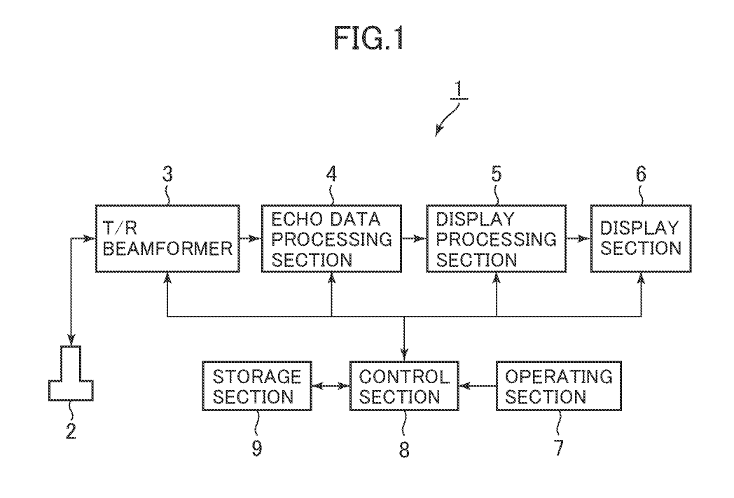

Now an embodiment of the present invention will be described. An ultrasonic diagnostic apparatus 1 shown in FIG. 1 comprises an ultrasonic probe 2, a transmission/reception (T/R) beamformer 3, an echo data processing section 4, a display processing section 5, a display section 6, an operating section 7, a control section 8, and a storage section 9. The ultrasonic diagnostic apparatus 1 has a configuration as a computer.

The ultrasonic probe 2 represents an exemplary embodiment of the ultrasonic probe in the present invention, which transmits ultrasound to biological tissue in a subject. By the ultrasonic probe 2, an ultrasonic pulse (push pulse) for generating shear waves in the biological tissue is transmitted. Also by the ultrasonic probe 2, an ultrasonic detecting pulse for detecting the shear waves is transmitted and echo signals thereof are received.

Moreover, by the ultrasonic probe 2, a B-mode imaging ultrasonic pulse for producing a B-mode image and a Doppler imaging ultrasonic pulse for producing a Doppler image are transmitted and echo signals thereof are received.

The T/R beamformer 3 drives the ultrasonic probe 2 based on control signals from the control section 8 to transmit the several kinds of ultrasonic beams with predetermined transmission parameters (a transmission control function). The T/R beamformer 3 also applies signal processing such as phased addition processing to the ultrasonic echo signals. The T/R beamformer 3 and control section 8 represent an exemplary embodiment of the transmission control section in the present invention. The transmission control function represents an exemplary embodiment of the transmission control function in the present invention.

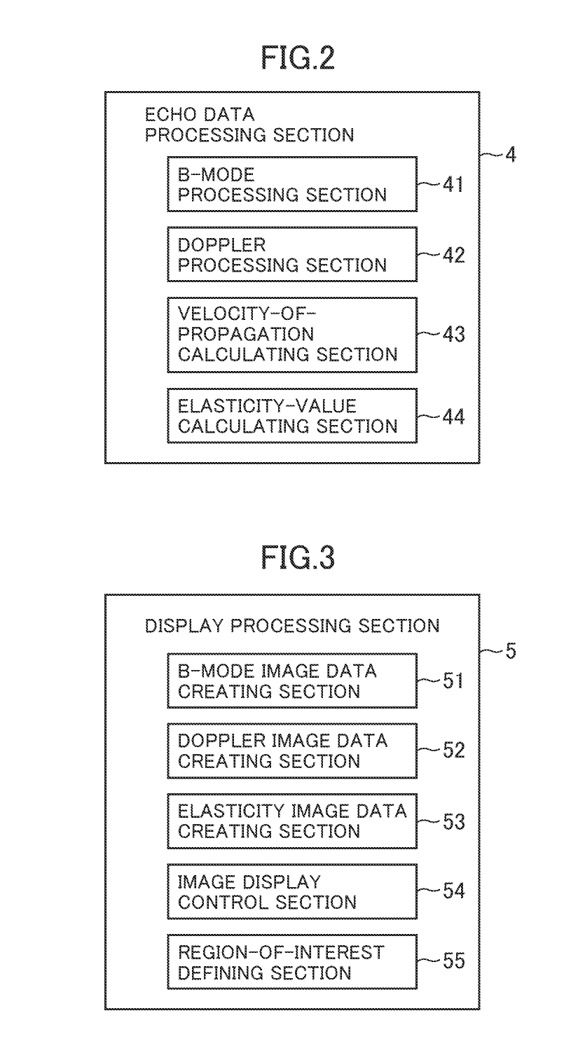

The echo data processing section 4 comprises a B-mode processing section 41, a Doppler processing section 4:2, a velocity-of-propagation calculating section 43, and an elasticity-value calculating section 44, as shown in FIG. 2. The B-mode processing section 41 applies B-mode processing such as logarithmic compression processing and envelope detection processing to echo data output from the T/R beamformer 3 to create B-mode data.

The Doppler processing section 42 applies Doppler processing to the echo data output from the T/R beamformer 3 to create Doppler data. The Doppler data is obtained for within a region of interest R, which will be discussed later. The Doppler processing includes quadrature detection processing and filtering.

The Doppler processing section 42 applies, for example, color Doppler processing for producing a color Doppler image in a color Doppler method. The color Doppler image is an image corresponding to the direction of blood flow and the magnitude of the velocity of the blood flow. The color Doppler image may include information on the variance. The Doppler processing section 42 may also apply power Doppler processing for producing a power Doppler image in a power Doppler method. The power Doppler image is an image corresponding to the value of the power representing the intensity of Doppler signals. The Doppler processing section 42 represents an exemplary embodiment of the Doppler processing section in the present invention. The function of creating Doppler data by the Doppler processing section 42 represents an exemplary embodiment of the Doppler processing function in the present invention.

The velocity-of-propagation calculating section 43 calculates a velocity of propagation of the shear waves based on echo data output from the T/R beamformer 3. The velocity-of-propagation calculating section 43 performs the calculation of the velocity of propagation based on quadrature detection-processed data of the echo data output from the T/R beamformer 3. The velocity of propagation is calculated based on the echo data from within the region of interest R, which will be discussed later. The velocity of propagation of shear waves within the region of interest R is thus calculated.

The velocity of shear waves in biological tissue varies depending upon elasticity of the biological tissue. Therefore, a velocity of propagation corresponding to elasticity of biological tissue can be obtained within the region of interest R.

When creation of Doppler data and calculation of the velocity of propagation are to be performed based on echo signals of common ultrasonic detecting pulses as will be discussed later, quadrature detection processing in the Doppler processing section 42 and that in the velocity-of-propagation calculating section 43 are combined in the echo data processing section 4. Specifically, creation of Doppler data and calculation of the velocity of propagation in a certain frame are achieved based on common echo data obtained by applying quadrature detection processing to echo data in that frame.

The elasticity-value calculating section 44 calculates an elasticity value of biological tissue to which a push pulse is transmitted based on the velocity of propagation. Details thereof will be discussed later. The velocity-of-propagation calculating section 43 and elasticity-value calculating section 44 represent an exemplary embodiment of the measurement-value calculating section in the present invention. The function of calculating a velocity of propagation by the velocity-of-propagation calculating section 43 and function of calculating an elasticity value by the elasticity-value calculating section 44 represent an exemplary embodiment of the function of calculating a measurement value in the present invention. The velocity of propagation and elasticity value represent an exemplary embodiment of the measurement value regarding elasticity of the biological tissue in the present invention.

It should be noted that only the velocity of propagation may be calculated without necessarily calculating the elasticity value. Data of the velocity of propagation or data of the elasticity value will be referred to herein as elasticity data.

The display processing section 5 comprises a B-mode image data creating section 51, a Doppler image data creating section 52, an elasticity image data creating section 53, an image display control section 54, and a region-of-interest defining section 55, as shown in FIG. 3. The B-mode image data creating section 51 scan-converts B-mode data by a scan converter to create B-mode image data. The Doppler image data creating section 52 scan-converts Doppler data by the scan converter to create Doppler image data. The elasticity image data creating section 53 scan-converts elasticity data by the scan converter to create elasticity image data. The Doppler image data creating section 52 represents an exemplary embodiment of the Doppler image data creating section in the present invention. The elasticity image data creating section 53 represents an exemplary embodiment of the elasticity image data creating section in the present invention.

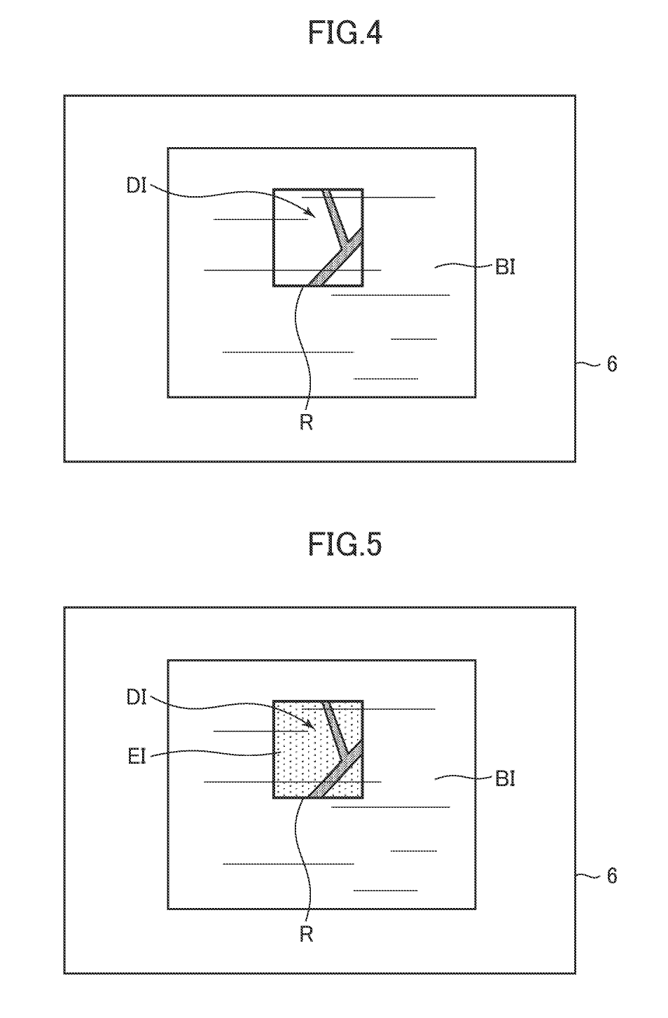

The image display control section 54 displays a B-mode image BI based on the B-mode image data in the display section 6. The image display control section 54 also displays a Doppler image DI based on the Doppler image data within a two-dimensional region of interest R defined in the B-mode image BI, as shown in FIG. 4.

Moreover, the image display control section 54 displays the Doppler image DI based on the Doppler image data and an elasticity image EI based on the elasticity image data within the two-dimensional region of interest R defined in the B-mode image BI, as shown in FIG. 5. The image display control section 54 represents an exemplary embodiment of the image display control section in the present invention.

More particularly, the image display control section 54 combines the B-mode image data with the elasticity image data to create combined image data, based on which it displays a combined image in the display section 6. The combined image is a semi-transparent color image through which the B-mode image BI in the background is allowed to pass. The color image is an image with colors depending upon the velocity of propagation or elasticity value, which is the elasticity image EI with colors depending upon elasticity of biological tissue. Moreover, the image display control section 54 displays the combined image further superimposed with the Doppler image DI. Thus, the elasticity image EI and Doppler image DI are displayed within the region of interest R. The Doppler image DI is a color Doppler image or a power Doppler image.

The image display control section 54 may produce a combined image by combining the image in which the B-mode image is superimposed with the color Doppler image, with the elasticity image for display.

The region of interest R is defined by the region-of-interest defining section 55. More particularly, the region-of-interest defining section 55 defines the region of interest R based on an input by the operator at the operating section 7. The region of interest R is a region for which shear waves are to be detected, and transmission/reception of the ultrasonic detecting pulse is performed in this region.

The display section 6 is an LCD (Liquid Crystal Display), an organic EL (Electro-Luminescence) display, or the like. The display section 6 represents an exemplary embodiment of the display section in the present invention.

The operating section 7 is configured to comprise a keyboard for allowing an operator to input a command and/or information, and further comprise a pointing device such as a trackball, and the like, although not particularly shown.

The control section 8 is a processor such as a CPU (Central Processing Unit). The control section 8 loads thereon a program stored in the storage section 9 and controls several sections in the ultrasonic diagnostic apparatus 1. For example, the control section 8 loads thereon a program stored in the storage section 9 and executes functions of the T/R beamformer 3, echo data processing section 4, and display processing section 5 by the loaded program.

The control section 8 may execute all of the functions of the T/R beamformer 3, all of the functions of the echo data processing section 4, and all of the functions of the display processing section 5 by the program, or execute only some of the functions by the program. In the case that the control section 8 executes only sonic of the functions, the remaining functions may be executed by hardware such as circuitry.

It should be noted that the functions of the T/R beamformer 3, echo data processing section 4, and display processing section 5 may be implemented by hardware such as circuitry.

The storage section 9 is a HDD (Hard Disk Drive), semiconductor memory such as RAM (Random Access Memory) and/or ROM (Read-Only Memory), and the like.

The ultrasonic diagnostic apparatus 1 may have all of the HDD, RAM, and ROM as the storage section 9. The storage section 9 may also be any portable storage medium such as a CD (Compact Disk) or a DVD (Digital Versatile Disk).

The program executed by the control section 8 is stored in a non-transitory storage medium, such as a HDD or ROM, constituting the storage section 9. The program may also be stored in any non-transitory portable storage medium, such as a CD or a DVD, constituting the storage section 9.

The storage section 9 may have the B-mode data, Doppler data, data of the velocity of propagation, and data of the elasticity value stored therein. The storage section represents an exemplary embodiment of the storage section in the present invention.

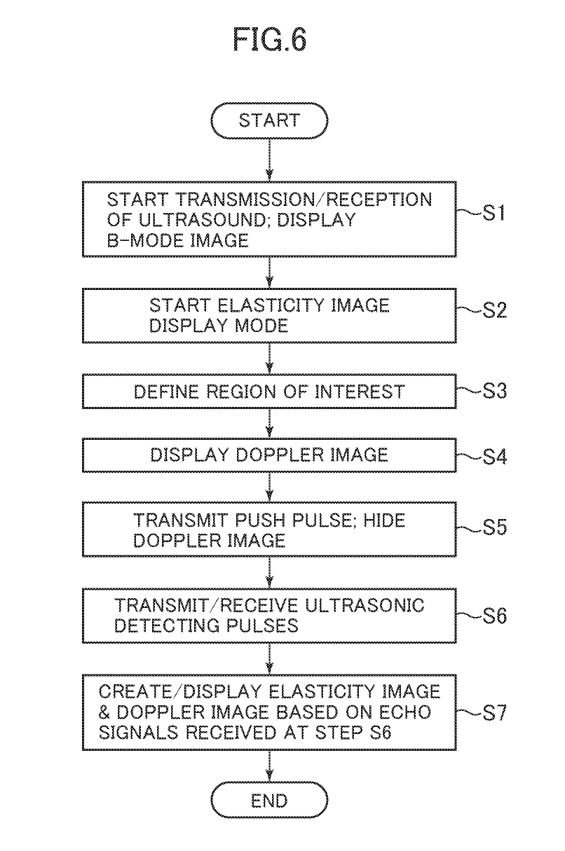

Next, an operation of the ultrasonic diagnostic apparatus 1 in the present embodiment will be described based on the flow chart in FIG. 6. The description here will address display of real-time B-mode, Doppler, and elasticity images.

First, at Step S1, a user starts ultrasound transmission/reception to/from biological tissue in a subject by the ultrasonic probe 2. The ultrasound transmission/reception is B-mode imaging ultrasound transmission/reception. Then, a B-mode image BI is displayed in the display section 6 at Step S1 here. The B-mode image BI is a real-time image and may be successively updated in the processing at the next Step S2 and thereafter.

Next, at Step S2, the user makes an input at the operating section 7 to start an elasticity image display mode for displaying an elasticity image EI. Next, at Step S3, the user defines a region of interest in the B-mode image BI. Once the region of interest R has been defined at Step S3, Doppler imaging ultrasound transmission/reception, in addition to the B-mode imaging ultrasound transmission/reception, is performed at Step S4. Then, a Doppler image DI is displayed at Step S4 here based on echo data obtained by the Doppler imaging ultrasound transmission/reception, as shown in FIG. 4 described above.

Once the Doppler image DI has been displayed at Step S4, the user may move the region of interest R using the operating section 7 to a region not including blood flow.

Next, at Step S5, the user makes an input at the operating section 7 to transmit a push pulse. Thus, a push pulse is transmitted from the ultrasonic probe 2. The push pulse is transmitted to, for example, the outside of the region of interest R and to the vicinity of one edge of the region of interest R in a lateral direction (X direction).

Moreover, the image display control section 54 turns the Doppler image DI displayed at Step S4 into a hidden state at Step S5 here. The Doppler image DI, however, may be in a display state at Step S5 here. In the latter case, once a new Doppler image has been produced at Step S7 described below, the Doppler image produced at Step S7 may be displayed in place of the Doppler image displayed at Step S4.

Next, at Step S6, an ultrasonic detecting pulse for detecting shear waves generated in the biological tissue by the push pulse transmitted at Step S5 is transmitted and echo signals thereof are received. The ultrasonic detecting pulse is transmitted a plurality of number of times at required transmission time intervals for each of a plurality of acoustic lines within the region of interest R, and echo signals thereof are received.

Next, at Step S7, an elasticity image EI and a Doppler image DI are produced for display based on the echo signals of the ultrasonic detecting pulse received at Step S6. Therefore, after transmission of the push pulse, Doppler imaging ultrasound transmission/reception is not performed separately from ultrasonic detecting pulse transmission/reception. However, the B-mode imaging ultrasound transmission/reception may be performed separately from the ultrasonic detecting pulse transmission/reception.

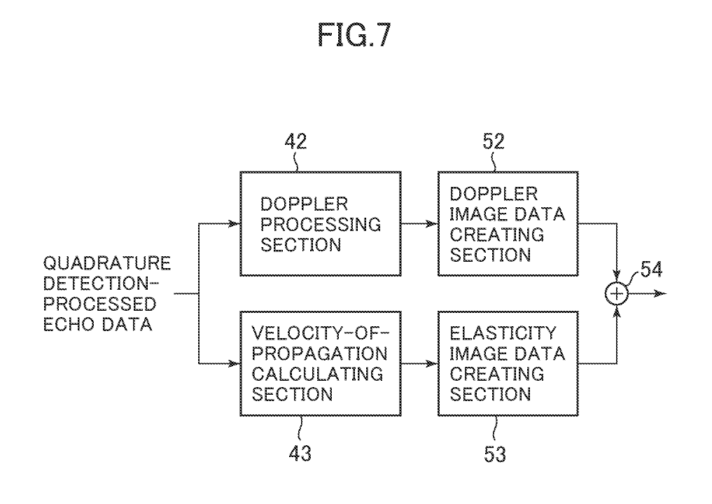

Now production of the elasticity image EI and Doppler image DI based on echo signals of the ultrasonic detecting pulse will be particularly described. FIG. 7 is a block diagram for explaining the processing at Step S7. The Doppler processing section 42 and velocity-of-propagation calculating section 43 perform processing on common quadrature detection-processed echo data. Then, based on Doppler data created by the Doppler processing section 42, the Doppler image data creating section 52 creates Doppler image data. Moreover, based on elasticity data (data of the velocity of propagation) created by the velocity-of-propagation calculating section 43, the elasticity image data creating section 53 creates elasticity image data. Although not shown in FIG. 7, the elasticity-value calculating section 44 may create an elasticity value based on the velocity of propagation to create the elasticity image data based on data of the elasticity value. The Doppler image data and elasticity image data are combined together by the image display control section 54, and an image in which the elasticity image EI is superimposed with the Doppler image DI is produced for display within the region of interest R, as shown in FIG. 5. The region of interest R is a region of interest defined in the B-mode image BI, and processing for the display section 6 by the image display control section 54 is similar to that described earlier. It should be noted that the B-mode processing section 41 and B-mode image data creating section 51 are omitted in FIG. 7.

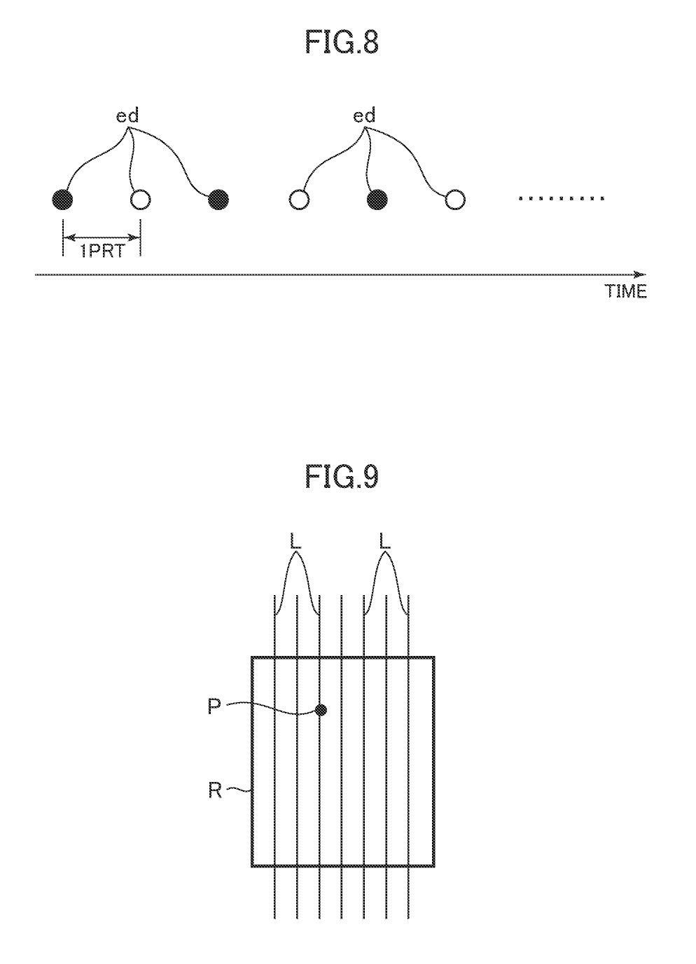

The processing at Step S7 will be described in more detail. FIG. 8 schematically shows echo data ed of the ultrasonic detecting pulse. It is assumed that the echo data ed is quadrature detection-processed data. The echo data ed are data obtained by transmitting a plurality of ultrasonic detecting pulses at required time intervals in one of a plurality of acoustic lines L within the region of interest R, as shown in FIG. 9. The echo data ed are also data obtained at a point P on one of the plurality of acoustic lines, wherein the point P is a point corresponding to one pixel in the elasticity image EI.

Since an ultrasonic detecting pulse is transmitted/received for one acoustic line a plurality of number of times as described above, a plurality of elements of echo data ed are obtained at one point on one acoustic line. In FIG. 8, the horizontal axis represents time, wherein the echo data ed more on the right side represents newer data. Each of the intervals of the plurality of elements of echo data ed represents a transmission time interval for the ultrasonic detecting pulse, i.e., 1 PRT (Pulse Repetition Time).

At Step S7 here, the velocity-of-propagation calculating section 43 calculates a velocity of propagation of shear waves detected by the echo data ed. The velocity of propagation is a velocity of propagation at the point P. The velocity-of-propagation calculating section 43 calculates the velocity of propagation at points other than the point P as well in the region of interest R in a similar manner. The elasticity-value calculating section 43 also calculates an elasticity value (Young's modulus (in Pa: Pascal)) based on the velocity of propagation. However, only the velocity of propagation may be calculated without calculating the elasticity value.

Moreover, the Doppler processing section 4 creates Doppler data based on the echo data ed. Again, the Doppler data is data at the point P. However, in general, the ultrasonic detecting pulse and Doppler imaging ultrasound have different transmission time intervals due to a difference in purpose between the purpose of detecting shear waves and the purpose of obtaining Doppler signals. In particular, the transmission time interval for the ultrasonic detecting pulse is shorter than that for the Doppler imaging ultrasound. Therefore, in the present embodiment, the transmission time interval for the ultrasonic detecting pulse is shorter than that for the Doppler imaging ultrasound before transmission of a push pulse.

Accordingly, the Doppler processing section 4:2 creates the Doppler data based on echo data ed for those of the plurality of ultrasonic detecting pulses in one acoustic line that have transmission time intervals longer than their transmission time intervals, i.e., time intervals longer than 1 PRT. For example, the Doppler processing section 42 may create Doppler data based on echo data ed (the echo data ed filled in black in FIG. 8) of ultrasonic detecting pulses having transmission time intervals twice those of the ultrasonic detecting pulses. The Doppler processing section 42 creates Doppler data at points other than the point P as well in the region of interest R in a similar manner.

The time interval for the echo data ed used for creating Doppler data is set to a time interval that provides Doppler data more accurately reflecting the blood flow information. The time interval may be set by default or by the user.

The elasticity image data creating section 53 creates elasticity image data based on the velocity of propagation calculated by the velocity-of-propagation calculating section 43 or the elasticity value calculated by the elasticity-value calculating section 44. The Doppler image data creating section 52 creates Doppler image data based on the Doppler data. The image display control section 54 then displays an image in which an elasticity image EI based on the elasticity image data is superimposed with a Doppler image DI based on the Doppler image data in the display section 6, as shown in FIG. 5 described above.

Steps S5 to S7 described above represent processing for displaying an elasticity image in one frame, and when the frame for the elasticity image is to be updated, the processing at Steps S5 to S7 is performed again.

Thus, the Doppler image DI is displayed in the region of interest R defined in the B-mode image BI, whereby the user can confirm whether a blood vessel exists within the region of interest R in which the elasticity image EI is displayed. Moreover, the elasticity image EI and Doppler image DI are displayed in the region of interest R, whereby the user can find out positional correspondence between a suspected lesion in the elasticity image and the position and distribution of a blood vessel, or find out in which direction blood flow lies with respect to the suspected lesion in the elasticity image.

Moreover, after a push pulse has been transmitted, Doppler data, as well as elasticity data (data of the velocity of propagation or data of the elasticity value), is created based on echo signals of the ultrasonic detecting pulse for detecting shear waves. Thus, since no transmission/reception for Doppler imaging ultrasound is performed aside from transmission/reception for ultrasonic detecting pulses, a Doppler image DI may be displayed along with an elasticity image EI without lowering the frame rate.

Furthermore, since Doppler data and elasticity data are created based on common echo signals, a Doppler image DI and an elasticity image EI at the same temporal phase may be displayed.

In addition, Doppler data is created based on echo data for those of a plurality of ultrasonic detecting pulses on one acoustic line that have longer time intervals than 1 PRT, whereby Doppler data more accurately reflecting blood flow information can be obtained.

While the present invention has been described with reference to the embodiment, it will be easily recognized that the present invention may be practiced with several modifications without changing the spirit and scope thereof. For example, while the Doppler image DI and elasticity image EI are displayed together in the embodiment described above, only one image may be switchably displayed. In this case, the image display control section 54 may switchably display the Doppler image DI and elasticity image EI based on, for example, an input by a user at the operating section 7.

Moreover, the image display control section 54 may display in the display section 6 a B-mode image, a Doppler image, and an elasticity image based on B-mode data, Doppler data and elasticity data (data of the velocity of propagation or data of the elasticity value) stored in the storage section 9, instead of real-time images.

Furthermore, the Doppler image does not have to be displayed before a push pulse is transmitted.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An ultrasonic diagnostic apparatus comprising:
a transmission control section for controlling transmission of an ultrasonic push pulse to biological tissue in a subject, and transmission of an ultrasonic detecting pulse for detecting shear waves generated in said biological tissue by said push pulse;
a measurement-value calculating section for calculating a measurement value regarding elasticity of said biological tissue based on echo signals of said ultrasonic detecting pulse; and
a Doppler processing section for creating Doppler data based on said echo signals of said ultrasonic detecting pulse.

2. The ultrasonic diagnostic apparatus as recited in claim 1, wherein:
said transmission control section causes an ultrasonic probe to transmit a plurality of said ultrasonic detecting pulses per acoustic line at required time intervals, and
said Doppler processing section creates said Doppler data based on echo signals of ultrasonic detecting pulses having longer time intervals than said required time intervals among the plurality of said ultrasonic detecting pulses.

3. The ultrasonic diagnostic apparatus as recited in claim 1, wherein:
calculation of said measurement value by said measurement-value calculating section and creation of said Doppler data by said Doppler processing section are performed based on common data obtained by applying quadrature detection processing to said echo signals of said ultrasonic detecting pulse.

4. The ultrasonic diagnostic apparatus as recited in claim 2, wherein:
calculation of said measurement value by said measurement-value calculating section and creation of said Doppler data by said Doppler processing section are performed based on common data obtained by applying quadrature detection processing to said echo signals of said ultrasonic detecting pulse.

5. The ultrasonic diagnostic apparatus as recited in claim 1, further comprising:
an elasticity image data creating section for creating elasticity image data based on the measurement value calculated by said measurement-value calculating section; and
a Doppler image data creating section for creating Doppler image data based on the Doppler data created by said Doppler processing section.

6. The ultrasonic diagnostic apparatus as recited in claim 2, further comprising:
an elasticity image data creating section for creating elasticity image data based on the measurement value calculated by said measurement-value calculating section; and
a Doppler image data creating section for creating Doppler image data based on the Doppler data created by said Doppler processing section.

7. The ultrasonic diagnostic apparatus as recited in claim 3, further comprising:
an elasticity image data creating section for creating elasticity image data based on the measurement value calculated by said measurement-value calculating section; and
a Doppler image data creating section for creating Doppler image data based on the Doppler data created by said Doppler processing section.

8. The ultrasonic diagnostic apparatus as recited in claim 4, further comprising:
an elasticity image data creating section for creating elasticity image data based on the measurement value calculated by said measurement-value calculating section; and
a Doppler image data creating section for creating Doppler image data based on the Doppler data created by said Doppler processing section.

9. The ultrasonic diagnostic apparatus as recited in claim 1, further comprising:
an image display control section for displaying a Doppler image based on said Doppler image data in a display section along with an elasticity image based on said elasticity image data.

10. The ultrasonic diagnostic apparatus as recited in claim 2, further comprising: an image display control section for displaying a Doppler image based on said Doppler image data in a display section along with an elasticity image based on said elasticity image data.

11. The ultrasonic diagnostic apparatus as recited in claim 3, further comprising: an image display control section for displaying a Doppler image based on said Doppler image data in a display section along with an elasticity image based on said elasticity image data.

12. The ultrasonic diagnostic apparatus as recited in claim 4, further comprising: an image display control section for displaying a Doppler image based on said Doppler image data in a display section along with an elasticity image based on said elasticity image data.

13. The ultrasonic diagnostic apparatus as recited in claim 1, further comprising: an image display control section for displaying an elasticity image based on said elasticity image data or a Doppler image based on said Doppler image data switchably in a display section.

14. The ultrasonic diagnostic apparatus as recited in claim 2, further comprising: an image display control section for displaying an elasticity image based on said elasticity image data or a Doppler image based on said Doppler image data switchably in a display section.

15. The ultrasonic diagnostic apparatus as recited in claim 3, further comprising: an image display control section for displaying an elasticity image based on said elasticity image data or a Doppler image based on said Doppler image data switchably in a display section.

16. The ultrasonic diagnostic apparatus as recited in claim 9, further comprising:
a storage section for storing therein data of the measurement value calculated by said measurement-value calculating section and the Doppler data created by said Doppler processing section, wherein
said image display control section displays an elasticity image based on the data of the measurement value and a Doppler image based on said Doppler data in said display section, said data of the measurement value and said Doppler data being stored in said storage section.

17. The ultrasonic diagnostic apparatus as recited in claim 11, further comprising:
a storage section for storing therein data of the measurement value calculated by said measurement-value calculating section and the Doppler data created by said Doppler processing section, wherein
said image display control section displays an elasticity image based on the data of the measurement value and a Doppler image based on said Doppler data in said display section, said data of the measurement value and said Doppler data being stored in said storage section.

18. The ultrasonic diagnostic apparatus as recited in claim 1, wherein: the Doppler data is color Doppler data according to a color Doppler technique or power Doppler data according to a power Doppler technique.

19. A method of measuring elasticity with an ultrasonic diagnostic apparatus, the method comprising:
controlling, with a transmission control section, a transmission of an ultrasonic push pulse to biological tissue in a subject;
controlling, with the transmission control section, transmission of an ultrasonic detecting pulse for detecting shear waves generated in said biological tissue by said push pulse;
calculating, with a measurement-value calculating section, a measurement value regarding elasticity of said biological tissue based on echo signals of said ultrasonic detecting pulse;
creating, with a Doppler processing section, Doppler data based on said echo signals of said ultrasonic detecting pulse; and
displaying an image based on the Doppler data on a display section.
